(19) United States
(12) Patent Application Publication
(10) Pub. No.: US 2007/0072210 A1
(43) Pub. Date: Mar. 29, 2007

(54) USE OF BRCA1-ASSOCIATED PROTEIN TO TREAT AND SCREEN FOR DNA DAMAGE AND TO IDENTIFY THERAPEUTICS THAT PROMOTE A DNA DAMAGE RESPONSE

(76) Inventors: Toru OUCHI, Wilmette, IL (US); Jason A. AGLIPAY, New York, NY (US)

Correspondence Address: NIXON PEABODY LLP - PATENT GROUP, CLINTON SQUARE, P.O. BOX 31051, ROCHESTER, NY, 14603-1051, US

(21) Appl. No.: 11/464,164

(22) Filed: Aug. 11, 2006

Related U.S. Application Data
(60) Provisional application No. 60/707,430, filed on Aug. 11, 2005.

(51) Int. Cl. C12Q 1/68; G01N 33/574

(52) U.S. Cl. 435/6; 435/7.23

(57) ABSTRACT

The present invention relates to the use of a BAAT1 protein to screen for potential therapeutics that promote a DNA damage response or treat breast cancer. The present invention also relates to a method of treating DNA damage or breast cancer in a subject by administering a compound that increases phosphorylation levels of ataxia telangiectasia mutated (ATM) under conditions effective to treat DNA damage. The present invention also relates to a method of ascertaining changes in DNA damage status in a subject. The present invention also relates to a method of detecting whether a subject is susceptible to breast cancer.

This application claims the benefit of U.S. Provisional Patent Application Ser. No. 60/707,430, filed Aug. 11, 2005, which is hereby incorporated by reference in its entirety.

The subject matter of this application was made with support from the United States Government under NCI Grant Nos. CA79892 and CA90631. The U.S. Government may have certain rights.

### FIELD OF THE INVENTION

The present invention relates to the use of a BRCA1-associated protein, BAAT1, to treat and screen for DNA damage in a subject, and to screen for potential therapeutics that promote a DNA damage response.

### BACKGROUND OF THE INVENTION

Breast cancer is the most common cancer and the second-leading cause of cancer mortality in women, with approximately one in nine being affected in their lifetime (Alberg et al., “Epidemiology, Prevention, and Early Detection of Breast Cancer,” Curr Opin Oncol 9:505-511 (1997)). Inheritance in breast cancer-families follows the classic Mendelian pattern of autosomal dominant transmission, with 50% of carriers' children inheriting breast caner susceptibility gene 1 (“BRCA1”) mutations. This inheritance pattern, as well as loss of heterozygosity (“LOH”) studies in tumors from affected members of BRCA1-linked families, supports the hypothesis that BRCA1 fits the model of a classic tumor suppressor gene, with loss of the normal allele in the tumors of all informative cases (Chamberlain et al., “BRCA1 Maps Proximal to D17S579 on Chromosome 17q21 by Genetic Analysis.” Am J Human Genet 52:792-798 (1993)). Female mutation carriers are estimated to have an 85% lifetime risk of breast cancer (Easton et al., “Genetic Linkage Analysis in Familial Breast and Ovarian Cancer: Results from 214 Families. The Breast Cancer Linkage Consortium.” Am J Human Genet 52:678-701 (1993)) and a 40% to 50% risk of ovarian cancer (Easton et al., “Breast and Ovarian Cancer Incidence in BRCA1 Mutation Carriers.” Am J Human Genet 56: 265-271 (1994)).

BRCA1, first identified as a breast cancer susceptibility gene, encodes an 1863 amino acid protein with an N-terminal RING finger domain and a C-terminal acidic domain termed the BRCT (Miki et al., “A Strong Candidate for the Breast and Ovarian Cancer Susceptibility Gene BRCA1.” Science 266:66-71 (1994)). Mutations in both alleles of BRCA1 greatly increase the risk of breast and ovarian cancer, identifying this gene as a tumor suppressor. Gene disruption experiments in mice result in early embryonic lethality and have therefore provided no information regarding BRCA1 function in adult animals (Gowen et al., “BRCA1 Deficiency Results in Early Embryonic Lethality Characterized by Neuroepithelial Abnormalities.” Nat Genet 12:191-194 (1996); Hakem et al., “The Tumor Suppressor Gene BRCA1 is Required for Embryonic Cellular Proliferation in the Mouse.” Cell 85:1009-1023 (1996); Liu et al, “Inactivation of the Mouse BRCA1 Gene Leads to Failure in the Morphogenesis of the Egg Cylinder in Early Postimplantation Development.” Genes Dev 10:1835-1843 (1996)). Mice resulting from conditional knockout showed immature mammary development and formed tumors after long latency with p53 mutation (Wu et al., “Conditional Mutation of BRCA1 in Mammary Epithelial Cells Results in Blunted Ductal Morphogenesis and Tumour Formation.” Nat Genet 22:37-43 (1999)).

The BRCA1 protein may act at a number of points in nuclear function and in growth control (Aglipay et al., “A Member of the Pyrin Family, IFI16, is a Novel BRCA1-Associated Protein Involved in the p53-Mediated Apoptosis Pathway.” Oncogene 22:8931-8938 (2003); Ouchi et al., “Collaboration of Signal Transducer and Activator of Transcription 1 (STAT1) and BRCA1 in Differential Regulation of IFN-Gamma Target Genes.” Proc Natl Acad Sci USA 97:5208-5213 (2000); Ouchi et al., “BRCA1 Phosphorylation by Aurora-A in the Regulation of G2 to M Transition.” J Biol Chem 279:19643-19648 (2004)). For example, the immunofluorescence pattern of BRCA1 dramatically changes from discrete nuclear dots to a dispersed pattern when cells are treated with chemical compounds or IR, implying that BRCA1 is involved in a replication checkpoint after DNA damage (Aglipay et al., “A Member of the Pyrin Family, IFI16, is a Novel BRCA1-Associated Protein Involved in the p53-Mediated Apoptosis Pathway.” Oncogene 22:8931-8938 (2003); Scully et al., “Dynamic Changes of BRCA1 Subnuclear Location and Phosphorylation State are Initiated by DNA Damage.” Cell 90:425-435 (1997); Chiba et al., “Redistribution of BRCA1 Among Four Different Protein Complexes Following Replication Blockage.” J Biol Chem 276:38549-38554 (2001); Okada et al., “Cell Cycle Differences in DNA Damage-Induces BRCA1 Phosphorylation Affect its Subcellular Localization.” J Biol Chem 278:2015-2020 (2003)). More recently, it was found that BRCA1 plays a crucial role in activating caspase 3 under UV damage (Martin et al., “BRCA1 Phosphorylation Regulates Caspase-3 Activation in UV-Induced Apoptosis.” Cancer Res 65:10657-10662 (2005)).

An appropriate response to DNA damage is crucial for maintenance of genome stability. Several cellular proteins have been implicated in such processes, such as ATM/ATR protein Ser/Thr kinases, Mre11/Rad51/NBS1 (MRN) complex, Fanconi anemia proteins, and BRCA1, breast cancer tumor susceptible protein (Shiloh et al., “ATM and Related Protein Kinases: Safeguarding Genome Integrity.” Nat Rev Cancer 3:155-168 (2003); Venkitaraman et al., “Tracing the Network Connecting BRCA and Fanconi Anaemia Proteins.” Nat Rev Cancer 4:266-276 (2004); Jackson et al., “Sensing and Repairing DNA Double-Strand Breaks.” Carcinogenesis 23:687-692 (2002); D'Andrea et al., “Molecular Biology of Fanconi Anemia: Implications for Diagnosis and Therapy.” Blood 90:1725-1736 (1997)). In human and murine cells, ataxia telangectasia mutated (“ATM”) is required for early response to agents such as ionizing radiation (“IR”) that induce DNA double stand breaks (“DSBs”). Despite considerable overlap between the processes regulated by ATM and its relative ATR (ataxia telangiectasia and Rad3-related) cells that lack ATM are extremely sensitive to IR. Thus, ATM plays a unique and essential role in determining survival following IR.

In its unstimulated state, ATM is proposed to exist as a homodimer in which the kinase domain of one subunit faces the autophosphorylation of another (Bakkenist et al., “DNA Damage Activates ATM Through Intermolecular Autophosphorylation and Dimer Dissociation.” Nature 421:499-506 (2003)). Upon stimulation, the intermolecular phosphorylation site of the subunits promotes dissociation and the monomers are free to phosphorylate other substrates (Bakkenist et al., “DNA Damage Activates ATM Through Intermolecular Autophosphorylation and Dimer Dissociation.” Nature 421:499-506 (2003); Lee et al., “Direct Activation of the ATM Protein Kinase by the Mre11/Rad50/Nbs1 Complex.” Science 304:93-96 (2004); Lee et al., “ATM Activation by DNA Double-Strand Breaks Through the Mre11-Rad50-Nbs1 Complex.” Science 308:551-554 (2005)). Use of the phosphorylation-site-specific antibody shows that almost maximal ATM autophosphorylation occurs within minutes of IR, even following very low doses. Indeed, ATM autophosphorylation is readily detectable when as few as two or four DSBs are generated enzymatically by ectopic expression of the restriction enzyme I-SceI (Bakkenist et al., “DNA Damage Activates ATM Through Intermolecular Autophosphorylation and Dimer Dissociation.” Nature 421:499-506 (2003)). Exposure of cells to mildly hypotonic buffers or to chromatin-modifying drugs, treatments that do not induce DSBs, also leads to rapid and near-maximal autophosphorylation of ATM. Together, these data suggest that a change in chromatin structure, rather than the presence of DSBs per se, is the stimulus to ATM autophosphorylation (Bakkenist et al., “DNA Damage Activates ATM Through Intermolecular Autophosphorylation and Dimer Dissociation.” Nature 421:499-506 (2003)).

The present invention is directed to overcoming these and other deficiencies in the art.

### SUMMARY OF THE INVENTION

The present invention relates to a method of screening for potential therapeutics that promote a DNA damage response. This method involves providing a breast cancer susceptibility 1 (“BRCA1”)-associated protein (referred to herein as “BAAT1 protein”), an ataxia telangiectasia mutated (“ATM”), and a candidate compound. The BAAT1 protein, the ATM, and the candidate compound are combined in a test mixture under conditions suitable for the ATM to undergo phosphorylation. Whether the candidate compound increases phosphorylation levels of the ATM compared to when the candidate compound is not present in the test mixture is evaluated. A candidate compound that increases phosphorylation levels of ATM compared to when the candidate compound is not present in the test mixture is identified as a potential therapeutic that promotes a DNA damage response.

The present invention also relates to a method of treating DNA damage in a subject. This method involves administering a compound that increases phosphorylation levels of ATM under conditions effective to treat DNA damage.

The present invention also relates to a method of ascertaining changes in DNA damage status in a subject. This method involves providing a sample from the subject. ATM phosphorylation levels in the sample are determined. ATM phosphorylation levels in different samples taken from the subject at different times are compared to ascertain changes in DNA damage status in the subject.

The present invention also relates to a method of screening for potential therapeutics for treating breast cancer. This method involves providing a BAAT1 protein, a BRCA1 protein, and a candidate compound. The BAAT1 protein, the BRCA1 protein, and the candidate compound are combined in a test mixture under conditions suitable for the BAAT1 protein to bind to the BRCA1 protein. Whether the candidate compound increases binding of the BAAT1 protein to the BRCA1 protein is evaluated. A candidate that increases binding of the BAAT1 protein to the BRCA1 protein as a potential therapeutic for treating breast cancer is identified.

The present invention also relates to a method of treating breast cancer in a subject. This method involves administering a compound to the subject which is effective in promoting binding of BAAT1 protein to BRCA1 protein under conditions effective to treat breast cancer.

The present invention also relates to a method of detecting whether a subject is susceptible to breast cancer. This method involves providing a sample from the subject. The level of binding of BRCA1 protein to BAAT1 protein binding in the sample is determined. The level of binding of BRCA1 protein to BAAT1 protein in the sample is compared to a standard level of binding of BRCA1 protein to BAAT1 protein for an individual that does not have breast cancer and/or for an individual that does have breast cancer. Whether the subject is susceptible to breast cancer based on how the level of level of binding of BRCA1 protein to BAAT1 protein in the sample correlates to the standard level is identified.

### BRIEF DESCRIPTION OF THE DRAWINGS

FIGS. 1A-C illustrate how BAAT1 Interacts with BRCA1. FIG. 1A shows a northern blot analysis of BAAT1 expression in human tissues. FIG. 1B shows the identification of the interacting region between BAAT1 and BRCA1. Indicated regions of GST-tagged BRCA1 and FLAG-BAAT1 were transiently expressed in 293T cells. GST-BRCA1 fragments were precipitated with GSH beads and samples were immunoblotted with anti FLAG antibody. To determine the BRCA1 -binding region, FLAG-tagged forms of BAAT1 were expressed in 293T cells. After immunoprecipitation with anti BRCA1 21A1 antibody, samples were immunoblotted with FLAG antibody. GST-fusion proteins and FLAG-BAAT1 were immunoblotted with anti GST and anti FLAG antibodies, respectively, to confirm their expression. NS: non-specific signals. FIG. 1C shows immunoprecipitation and immunoblot analysis showing that endogenous BRCA1 and ATM can immunoprecipitate endogenous BAAT1 from HeLa cells. Two different anti BRCA1 antibodies (C-20 and 21A1) and anti ATM antibodies (N-17 and 2C1) were used for the assay.

FIGS. 2A-B show the BAAT1 association with ATM. FIG. 2A shows treatment of NMEs with IR induces association of BAAT1 with ATM or BRCA1. After immunoprecipitation of ATM, samples were immunoblotted with BAAT1. BRCA1 was immunoprecipitated 1 h after IR treatment. Treatment of Tubulin was immunoblotted as a loading control. FIG. 2B illustrates how the localization of BAAT1 with DSBs was shown with co-immunostaining with γH2AX in NMEs after IR treatment (1 h). Specificity of anti BAAT1 antibody was examined in NMEs cells in which BAAT1 had been knocked down by siRNA (see FIG. 4A). A scale bar: 10 μm.

FIGS. 3A-C show the immunocytochemical analysis of BAAT1, BRCA1 and ATM. FIG. 3A shows how NMEs were treated with IR and localization of BAAT1, BRCA1 (21A1 antibody) and the overlapped foci were analyzed by confocal microscopy after 1 h. FIG. 3B shows SNU251 cells were infected with adenovirus BRCA1 or control LacZ virus. Cells were analyzed for immunoblot or immunocytochemistry with anti BRCA1 C20 antibody to confirm the expression. FIG. 3C shows SNU251 cells were infected with the indicated virus and treated with IR (5 h). Localization of BAAT1, ATM, and the overlapped foci is shown. A scale bar: 10 μm.

FIG. 4 A-C show down-regulation of BAAT1 via siRNA in NMEs. FIG. 4A shows BAAT1 levels in isolated clones of NMEs stably transfected with BAAT1 siRNA (left). BAAT1 levels in U2OS cells transiently transfected with BAAT1 siRNA for 48 h (reight). FIG. 4B (left) shows the phosphorylation of ATM and Chk2 in clone #1 after treatment with IR (5Gy, 30 min to 12 h). Levels of p53 were also studied in these cells. Phosphorylation of ATM and Chk2 in U2OS cells transiently transfected with BAAT1 siRNA (IR, 5Gy, 1 h) (right). FIG. 4C (left) shows ATM phosphorylation of Ser1981 in NMEs, HCC1937 and SNU251 cells after IR treatment. Cells were irradiated with 3Gy or 5Gy of IR and immunoblotted after 1 h. SNU251 cells were infected with control or BRCA1 adenovirus for 48 h and treated with IR (5Gy) (right). After 1 h, cell lysates were immunoblotted with the indicated antibody.

FIG. 5 shows annexin V analysis of NMEs stably transfected with BAAT1 siRNA and U2OS cells transiently transfected with BAAT1 siRNA. Cells were treated with IR (5Gy) and apoptotic cells were studied by Annexin V staining after 24 h.

FIG. 6 shows decreased phosphorylation of ATMSer1981, γH2AX and NBS1 in BAAT1 knockdown cells after IR treatment. Immunostaining (green) of Ser1981-phosphorylated ATM (a-h), γH2AX (i-p) and Ser343-phosphorylated NBS1 (q-x) were studied after 1 h using NMEs transfected with control- or BAAT1-siRNA. A scale bar: 10 μm.

FIGS. 7A-C show the protection of ATM Ser1981 phosphorylation by BAAT. FIG. 7A (left) shows okadaic acid treatment restored ATM Ser1981 phosphorylation after IR. Control or BAAT1-siRNA NMEs were pre-incubated with okadaic acid (0.5 mM, 3 h) and treated with IR (5Gy). Cells were collected after 30 min and subjected to immunoblot analysis with indicated antibodies. Interaction of ATM and PP2A was examined in control- or BAAT1-siRNA cells (Right). FIG. 7B shows HEK293T cells were transfected with HA-tagged BAAT1 for 48 h. One hour after IR treatment, cells were lysed and incubated with PP2A. Samples were immunoblotted with the indicated antibodies. FIG. 7C (top) shows the in vitro PP2A assay of phosphorylated ATM. NMEs were treated with IR and lysed after 1 h. After immunoprecipitation of ATM, optimal concentration of PP2A was determined by in vitro phosphatase assay. Similarly, ATM phosphorylation was studied by in vitro PP2A assay using 0.1 unit of PP2A. Purified BAAT1 (100 to 800 ng) was simultaneously added to the samples (lower panels).

FIG. 8 is an alignment of the amino acid sequences of BAAT1 in human (hBAAT1) and mouse (mBAAT1). Conserved amino acids are shown in bold letters. Underlined peptides were used to generate rabbit polyclonal antibodies.

### DETAILED DESCRIPTION OF THE INVENTION

The present invention relates to a method of screening for potential therapeutics that promote a DNA damage response. This method involves providing a BAAT1 protein, an ataxia telangiectasia mutated (“ATM”), and a candidate compound. The BAAT1 protein, the ATM, and the candidate compound are combined in a test mixture under conditions suitable for the ATM to undergo phosphorylation. Whether the candidate compound increases phosphorylation levels of the ATM compared to when the candidate compound is not present in the test mixture is evaluated. A candidate compound that increases phosphorylation levels of ATM compared to when the candidate compound is not present in the test mixture is identified as a potential therapeutic that promotes a DNA damage response.

As used herein, the term “DNA damage” includes double strand breaks in the DNA. DNA damage is induced by a variety of agents such as ultraviolet light, X rays, free radicals, methylating agents and other mutagenic compounds. These agents may cause damage to the DNA that comprises the genetic code of an organism and cause mutations in genes. In microorganisms such mutations may lead to the evolution of new undesirable strains of the microorganism. For instance, antibiotic or herbicide resistant bacteria may arise. In animals these mutations can lead to carcinogenesis or may damage the gametes to give rise to congenital defects in offspring. These DNA damaging agents may chemically modify the nucleotides that comprise DNA and may also break the phosphodiester bonds that link the nucleotides or disrupt association between bases (T-A or C-G).

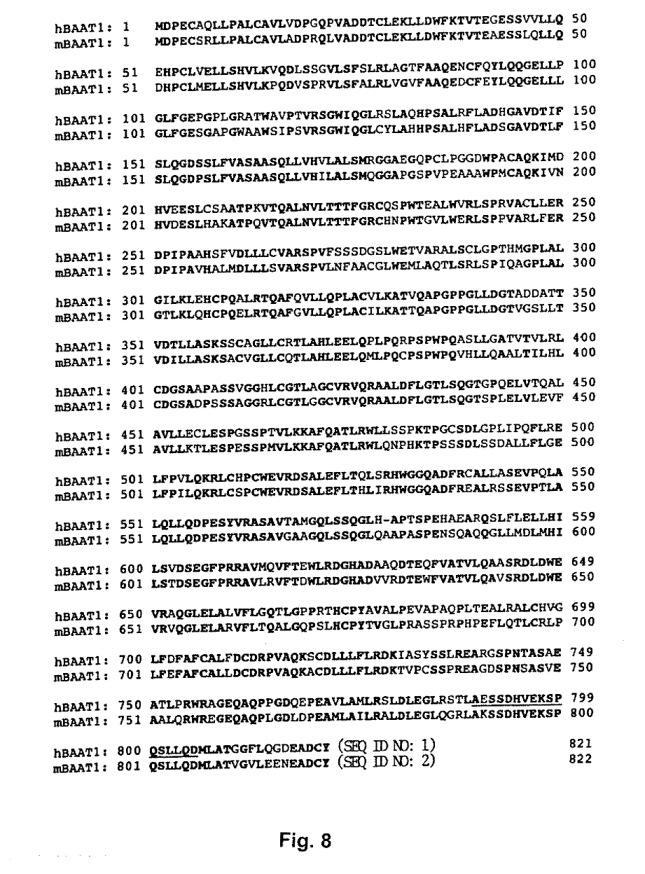

BAAT1 proteins suitable for use in the present invention can include, for example, human BAAT1 (hBAAT1) (e.g., SEQ ID NO:1) (see FIG. 8) and mouse BAAT1 (mBAAT1) (e.g., SEQ ID NO:2) (see FIG. 8), as well as homologous proteins known from other species, including, for example, from species such as Canis familiaris, Dania rerio, Gallus gallus, Mus musculus, Pan troglodytes, Rattus norvegicus, Xenopus tropicalis, Tetraodon nigroviridis, and Fugu rubripes. These homologous proteins are readily identifiable by those of ordinary skill in the art (e.g., by using protein databases such as NCBI and Ensembl Database).

Various methods and conditions well known in the art can be used to combine the BAAT1 protein, the ATM, and the candidate compound so as to provide suitable conditions for the ATM to undergo phosphorylation.

In a particular embodiment, the test mixture can further include a BRCA1 gene. Suitable BRCA1 genes are readily known by those of ordinary skill in the art, and can be found in various gene databases (e.g., GenBank, NCBI, EMBL).

Suitable ways of evaluating whether the candidate compound increases phosphorylation of ATM is to examine phosphorylation of ATM at serine¹⁹⁸¹. Increased phosphorylation of ATM is effective in identifying a candidate compound as having potential as a therapeutic that promotes a DNA damage response.

The present invention also relates to a method of treating DNA damage in a subject. This method involves administering a compound that increases phosphorylation levels of ATM under conditions effective to treat DNA damage.

As used herein, the term “subject” is meant to include mammals, and more particularly, humans. Suitable compounds that can be used in this method can include, without limitation, those compounds identified as being effective in increasing phosphorylation levels of ATM (according to other methods of the present invention, as described herein).

Administering the compound to the subject in accordance with the methods of treatment (described herein) can be achieved using processes and conditions well known in the art. Suitable examples of such administering can include administering the compound orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The solid unit dosage forms can be of the conventional type. The solid form can be a capsule, such as an ordinary gelatin type containing a therapeutic compound (including compounds that are effective in increasing phosphorylation levels of ATM and/or effective in promoting binding of a BAAT1 protein to a BRCA1 protein) and a carrier, for example, lubricants and inert fillers such as, lactose, sucrose, or cornstarch. In another embodiment, these therapeutic compounds are tableted with conventional tablet bases such as lactose, sucrose, or cornstarch in combination with binders like acacia, cornstarch, or gelatin, disintegrating agents such as, cornstarch, potato starch, or alginic acid, and a lubricant like stearic acid or magnesium stearate.

In another one aspect, the drugs containing the therapeutic compounds of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, the drugs of the present invention may be incorporated with excipients and used in the form of tablets, capsules, elixirs, suspensions, syrups, and the like. In one aspect, such drugs should contain at least 0.1% of the therapeutic compound of the present invention. The percentage of the therapeutic compound in the drugs of the present invention may, of course, be varied and may conveniently be between about 2% to about 60% of the weight of the unit. The amount of active therapeutic compound in the drugs of the present invention is such that a suitable dosage will be obtained. As one example, drugs according to the present invention are prepared so that an oral dosage unit contains between about 1 and 250 mg of therapeutic compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

As described above, in one aspect of the present invention, the drugs containing the therapeutic compounds may be administered parenterally. Solutions or suspensions of the therapeutic compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

Drugs containing the therapeutic compounds of the present invention may also be administered directly to the airways in the form of an aerosol. For use as aerosols, the therapeutic compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The present invention also relates to a method of ascertaining changes in DNA damage status in a subject. This method involves providing a sample from the subject. ATM phosphorylation levels in the sample are determined. ATM phosphorylation levels in different samples taken from the subject at different times are compared to ascertain changes in DNA damage status in the subject.

A suitable “sample” from the subject can include, for example, a bodily fluid or tissue from the subject. Suitable bodily fluids can include, without limitation, urine, blood, sputum, serum, plasma, saliva, and cerebrospinal fluid. Suitable tissue can include any bodily tissue from a mammalian subject (e.g., by biopsy).

In a particular embodiment, determination of changes in DNA damage status can include providing a BAAT1 protein (as described to herein) and an ataxia telangiectasia mutated. The BAAT1 protein, the ataxia telangiectasia mutated, and the sample are combined in a test mixture under conditions suitable for the ataxia telangiectasia mutated to undergo phosphorylation. Thereafter, whether the phosphorylation level of the ataxia telangiectasia mutated has changed is evaluated. In a further embodiment, determining phosphorylation of ataxia telangiectasia mutated levels in the sample can involve examining phosphorylation of ataxia telangiectasia mutated at serine¹⁹⁸¹. The test mixture can further include, for example, a BRCA1 gene.

The present invention also relates to a method of screening for potential therapeutics for treating breast cancer. This method involves providing a BAAT1 protein, a BRCA1 protein, and a candidate compound. The BAAT1 protein, the BRCA1 protein, and the candidate compound are combined in a test mixture under conditions suitable for the BAAT1 protein to bind to the BRCA1 protein. Whether the candidate compound increases binding of the BAAT1 protein to the BRCA1 protein is evaluated. A candidate compound that increases binding of the BAAT1 protein to the BRCA1 protein is identified as a potential therapeutic for treating breast cancer. Suitable BAAT1 proteins, BRCA1 proteins, and candidate compounds include those previously described herein.

The present invention also relates to a method of treating breast cancer in a subject. This method involves administering a compound to the subject which is effective in promoting binding of BAAT1 protein to BRCA1 protein under conditions effective to treat breast cancer. Suitable BAAT1 proteins and BRCA1 proteins include those previously described herein.

The present invention also relates to a method of detecting whether a subject is susceptible to breast cancer. This method involves providing a sample from the subject. The level of binding of BRCA1 protein to BAAT1 protein binding in the sample is determined. The level of binding of BRCA1 protein to BAAT1 protein in the sample is compared to a standard level of binding of BRCA1 protein to BAAT1 protein for an individual that does not have breast cancer and/or for an individual that does have breast cancer. Whether the subject is susceptible to breast cancer based on how the level of level of binding of BRCA1 protein to BAAT1 protein in the sample correlates to the standard level is identified. Suitable BAAT1 proteins and BRCA1 proteins include those previously described herein.

In a particular embodiment, determining a level of binding of BRCA1 protein to BAAT1 protein binding in the sample can include, without limitation, the following steps: providing a BAAT1 protein and a BRCA1 protein, and combining these two proteins with the sample in a test mixture under conditions suitable for the BAAT1 protein and the BCRA1 to bind. The binding level of BRCA1 and BAAT1 in the test mixture is then evaluated. Suitable BAAT1 proteins, BRCA1 proteins, samples, and conditions include those previously described herein.

### EXAMPLES

The following examples are provided to illustrate embodiments of the present invention but are by no means intended to limit, in any way, the scope of the present invention.

### Materials and Methods for Examples 1-7

Identification and Cloning of BAAT1: The Cyto-Trap Vector Kit (Stratagene) was used to identify proteins that interact with the C-terminal region of BRCA1. A 0.6 kbp NcoI and NotI digested DNA fragment of the BRCA1 C-terminal domain containing amino acids 1650-1863 in BRCA1/pcDNA3 was sub-cloned into the pSOS vector and designated as bait. The hSOS/BRCT fusion construct was co-expressed in cdc25H yeast expressing the myristoylated target cDNA library derived from human thymus and allowed to incubate at 37° C. Plasmids from the yeast colonies that grew at 37° C. were isolated and the DNA sequenced for identification and cloning.

Cell Culture, Transfections, and Western Blotting: NMEs (184B5 cells), HeLa U2OS and 293T cells were grown in DMEM containing 10% fetal bovine serum and 100 U of penicillin-streptomycin/ml. SNU-251 cells were grown in RPMI medium containing 10% fetal bovine serum, and 100 U of penicillin-streptomycin and supplemented with 1 mM sodium pyruvate. For transfections, the calcium phosphate method was used except when stably expressing BAAT1 siRNA was generated while using the FUGENE6 reagent (Roche) according to the manufacturer's protocol. PP2A antibody was obtained from the Mt. Sinai School of Medicine.

Plasmids and Antibodies: IMAGE clone BC015632 in pOTB7 vector was purchased (Resgen). GST-BRCA1 constructs were previously described (11,12) or generated by PCR. The antibodies C20, Ab2 (BRCA1), 2C1 (ATM) and ATMSer1981-P were obtained from Santa Cruz, Calbiochem, GeneTex, Rockland, respectively. Anti-ATM (N-17) was obtained from Santa Cruz and anti BRCA1 (21A1) was described previously (Aglipay et al., “A Member of the Pyrin Family, IFI16, is a Novel BRCA1-Associated Protein Involved in the p53-Mediated Apoptosis Pathway.” Oncogene 22:8931-8938 (2003); Okada et al., “Cell Cycle Differences in DNA Damage-Induces BRCA1 Phosphorylation Affect its Subcellular Localization.” J Biol Chem 278:2015-2020 (2003), which are hereby incorporated by reference in their entirety). Anti-BAAT1 antibody was raised against synthesized peptide (AESSDHVEKSPQSLLQD) (SEQ ID NO:3) (Covance). Anti p53 antibody (DO1) was from Santa Cruz.

Co-Immunoprecipitation and GST Pull-Down Assays: HeLa or NMEs cells were lysed for 30 min in 500 ml of lysis buffer (50 mM Tris-HCl pH 7.5, 100 mM NaCl, 0.5% NP-40, 0.5% DOC, 5 mM EDTA, and protease inhibitors: 100 mg/ml PMSF, 2 mg/ml Aprotinin, 2 mg/ml Leupeptin). One milligram of total cell lysates was incubated with 10 mg/ml of ethidium bromide and the primary antibody for 3 h followed by incubation with either Protein A or Protein G agarose beads overnight at 4° C. The immunocomplexes were separated on SDS-PAGE gels and visualized by Western blotting. To map the interaction site of BAAT1 and BRCA1, 293T cells were co-transfected with both GST BRCA1 construct and FLAG-BAAT1. After pulldown with GSH beads, samples were separated in SDS-polyacrylamide gel and visualized by Western blotting.

Northern Blotting: Full-length BAAT1 and β-actin cDNAs were radiolabeled with radioactive α³²P dCTP according to the manufacturer's protocol (Ambion). The Human PolyA+RNA Northern Blot (12 tissues) Membrane (Origene Technologies) was blotted with the radiolabeled probes according to the manufacturer's protocol.

Purification of Baculoviral BAAT1: His-tagged full length BAAT1 was produced in Sf9 cells and purified by Ni-column using the same protocol described previously (Ouchi et al., “Collaboration of Signal Transducer and Activator of Transcription 1 (STAT1) and BRCA1 in Differential Regulation of IFN-Gamma Target Genes.” Proc Natl Acad Sci USA 97:5208-5213 (2000), which is hereby incorporated by reference in its entirety).

Immunocytochemical Analysis: Cells were grown in chamber slides (Fisher) and fixed in −20° C. 100% methanol for 10 min. After three 5 min washes in TBS, the cells were quenched with fresh 0.1 % sodium borohydride in PBS (0.58M sodium phosphate dibasic (Na₂HPO₄), 0.17M sodium phosphate monobasic (NaH₂PO₄), 0.68M NaCl, adjusted to pH 7.4) for 5 min. A blocking solution (10% Goat serum, 1% Bovine Serum Albumin, 0.02% NaN₃ made in PBS) was used prior to incubation with the primary antibodies BAAT1, BRCA1 24E6 (Aglipay et al., “A Member of the Pyrin Family, IFI16, is a Novel BRCA1-Associated Protein Involved in the p53-Mediated Apoptosis Pathway.” Oncogene 22:8931-8938 (2003), which is hereby incorporated by reference in its entirety), ATM AB2 (Oncogene Research), ATM pSer1981 (Rockland), pγH2AX (Upstate Signaling) and NBS1 pSer343 (Santa Cruz). All secondary antibodies used were species-specific fluorochrome-conjugated antibodies from Jackson Immunoresearch (Texas Red-X for mouse IgG and fluorescein isothiocyanate (FITC) for rabbit IgG), used at 1: 100 throughout the experiments). The cell nuclei were stained with DAPI stain (Sigma). The fluorescence patterns were detected by laser confocal microscopy (Zeiss LSM 510 Meta confocal microscope) and the data analyzed and collected using the LSM 510 program (version 3.2; Zeiss). To minimize the possibility of signal cross-talk, a multi-track configuration was used during data collection.

Establishment of siRNA-Expressing Cells: BAAT1-siRNA target sequence 5′-AACGGUCACUGAAGGAGA-3′ (SEQ ID NO:4) (Dharmacon) was subcloned into the BglII site of the pSUPER vector. The control pSUPER/LucsiRNA (5′-GTTACGCTGAGTACTTCGA-3′) (SEQ ID NO:5) (obtained from Mount Sinai School of Medicine). About 1×10⁶ of NMEs cells were transfected with pBabepuro vector (Osaka Bioscience Institute) at a 1:10 ratio and pSUPER/LucsiRNA control or pSUPER/BAAT1 siRNA. Cell colonies were isolated (puromycin. 2 μg/ml) and the down-regulation of BAAT1 expression analyzed by western blot. For the okadaic acid assay, cells were pre-incubated with okadaic acid (0.5 mM) for 3 h and treated with IR (5Gy). After 30 min, cell lysates were immunoblotted. For transient transfection of U2OS cells, double strand RNA of the same sequence above was introduced into the cells with oligofectamine (Invitrogen). After 48 h, cells were subjected to Annexin V staining or immunoblot analysis.

Annexin V/Propidium Iodide Staining and Trypan Blue Staining: Cells were washed with PBS and harvested by trypsinization and the concentration adjusted to 1×10⁶ cells/ml. Annexin V was detected using an Annexin V-FITC Apoptosis Detection Kit (Oncogene Research) as recommended by the manufacturer. For the trypan blue assay, cells were treated with IR and surviving cells were counted 24 h after staining them with trypan blue (0.4% w/v).

Phosphatase Protection Assay: Human embryonic kidney (“HEK”) 293T cells (2×10⁶) were transfected with HA-tagged BAAT1. After 48 h, cells were lysed with EBC buffer which does not contain phosphatase inhibitor. Twenty micro grams of cell lysates were incubated with PP2A (0.1 unit, Upstate Bio). For in vitro PP2A assay, ATM was immunoprecipitated from NMEs treated by IR (5Gy, 1 h). Samples were washed extensively by NET-N buffer and PP2A buffer (Upstate Bio), and resuspended in 20 μl of PP2A buffer. After incubation with purified BAAT1 for 15 min, 0.1 unit/sample of PP2A was added for 30 min at room temperature.

### Example 1

### Screening and Isolation of BRCT Domain-Interacting Proteins

Because the C-terminal region of BRCA1 contains transactivation activity (Chápman et al., “Transcriptional Activation by BRCA1,” Nature 382:678-679 (1996), which is hereby incorporated by reference in its entirety), it is difficult to isolate interacting proteins using the conventional yeast two-hybrid screen. A modified yeast two-hybrid screening was used with the amino acids 1650-1863 region of BRCA1 as bait. Four cDNA fragments were obtained that encode partial sequences of metalloproteinase, ribosomal protein L13, elongation factor G-like protein, and a C-terminal part of the uncharacterized C7ORF27 protein. Previously known BRCT-associated proteins such as CtIP and BACH1 were not isolated from this screening.

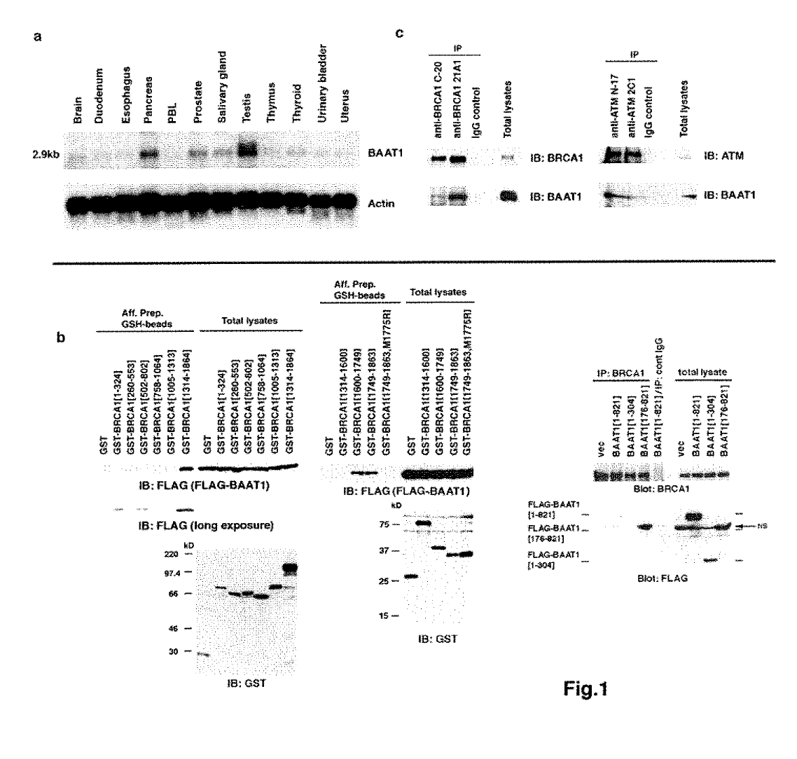

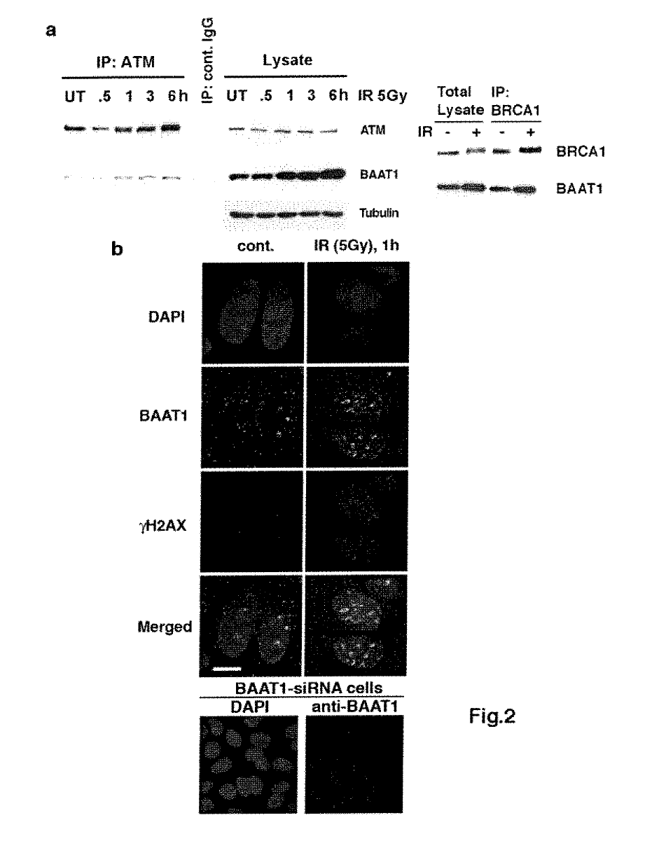

An isolated C7ORF27 gene encodes the open reading frame of 546 amino acids, which is a C-terminal part of the full-length cDNA registered in GenBank (accession number BC015632). The full-length protein consists of 821 amino acids of MW˜100 kDa which does not show significant homology or motifs such as a nuclear localizing signal to proteins registered in the database. The amino acid sequences of the human and mouse proteins (NCBI accession number BAC39362) share 73% identity. Homologous proteins of other species are known in the art, including, for example, Canis familiaris, Dania rerio, Gallus gallus, Mus musculus, Pan troglodytes, Rattus norvegicus, Xenopus tropicalis, Tetraodon nigroviridis, and Fugu rubripes (see Ensembl Database at www.ensembl.org). The protein was named BAAT1 (BRCA1-asssociated protein required for ATM activation-1) on the basis of its possible roles in the BRCA1/ATM pathway (described below). Northern blot analysis using multiple human tissues showed that a 2.9-kb transcript of BAAT1 is ubiquitously expressed, although slightly increased levels of mRNA are detected in testis and pancreas (FIG. 1A).

### Example 2

### BAAT1 Associates with BRCA1 and ATM

Interaction of C-terminal BRCA1 and BAAT1 was confirmed using FLAG-tagged BAAT1 and GST-tagged BRCA1 fragments co-expressed in 293T cells. After pulldown of the GST segments using glutathione beads, it was found that FLAG-BAAT1 preferentially binds to amino acids 1600-1749 (BRCT-N) and amino acids 1749-1863 (BRCT-C) of the BRCA1 protein (Botuyan et al., “Structural Basis of BACH1 Phosphopeptide Recognition by BRCA1 Tandem BRCT Domains,” Structure 12:1137-1146 (2004), which is hereby incorporated by reference in its entirety), consistent with the results of the yeast two-hybrid screen by which BAAT1 was isolated (FIG. 1B). Long exposure of the X-ray film showed that BRCA1 amino acids 1-324 and 502-802 weakly bind to BAAT1. Significantly, a breast cancer-associated mutant form of BRCT domains, Met1775Arg (M1775R), did not bind to BAAT1, suggesting that interaction of BRCA1 with BAAT1 is crucial for the tumor suppressive activity of BRCA1. The C-terminal BAAT1 (amino acids 176-821) showed stronger interaction with BRCA1 than the full length BAAT1 in this assay (FIG. 1B, Right panel). It has been shown that BRCA1 forms a large complex with cellular proteins termed BASC (BRCA1-associated genome surveillance complex), which include ATM (Cortez et al., “Requirement of ATM-Dependent Phosphorylation of BRCA1 in the DNA Damage Response to Double-Strand Breaks,” Science 286:1162-1166 (1999), which is hereby incorporated by reference in its entirety). Several lines of investigation have also demonstrated that BRCA1 is a substrate of ATM and is involved in the ATM/ATR (ATM-related) pathway (Gatei et al, “Role for ATM in DNA Damage-Induced Phosphorylation of BRCA1,” Cancer Res 60:3299-3304 (2000); Gatei et al., “Ataxia Telangiectasia Mutated (ATM) Kinase and ATM and Rad3 Related Kinase Mediate Phosphorylation of BRCA1 at Distinct and Overlapping Sites. In Vivo Assessment Using Phospho-Specific Antibodies (2001); Foray et al., “A Subset of ATM- and ATR-Dependent Phosphorylation Events Requires the BRCA1 Protein,” EMBO J 22:2860-2871 (2003); Xu et al., “Phosphorylation of Serine 1387 in BRCA1 is Specifically Required for the ATM-Mediated S-phase Checkpoint After Ionizing Irradiation,” Cancer Res 62:4588-4591 (2002), which are hereby incorporated by reference in their entirety). Co-immunoprecipitation of BAAT1 with BRCA1 was detected with two different anti BRCA1 antibodies (C-20 and 21A1), and physical interaction of BAAT1 with ATM was detected when ATM was immunoprecipitated with two different antibodies (N-17 and 2C1) from HeLa cells, suggesting that BAAT1 is involved in the DNA damage pathway regulated by BRCA1 and ATM (FIG. 1C).

### Example 3

### BAAT1 is Involved in IR Damage Pathway

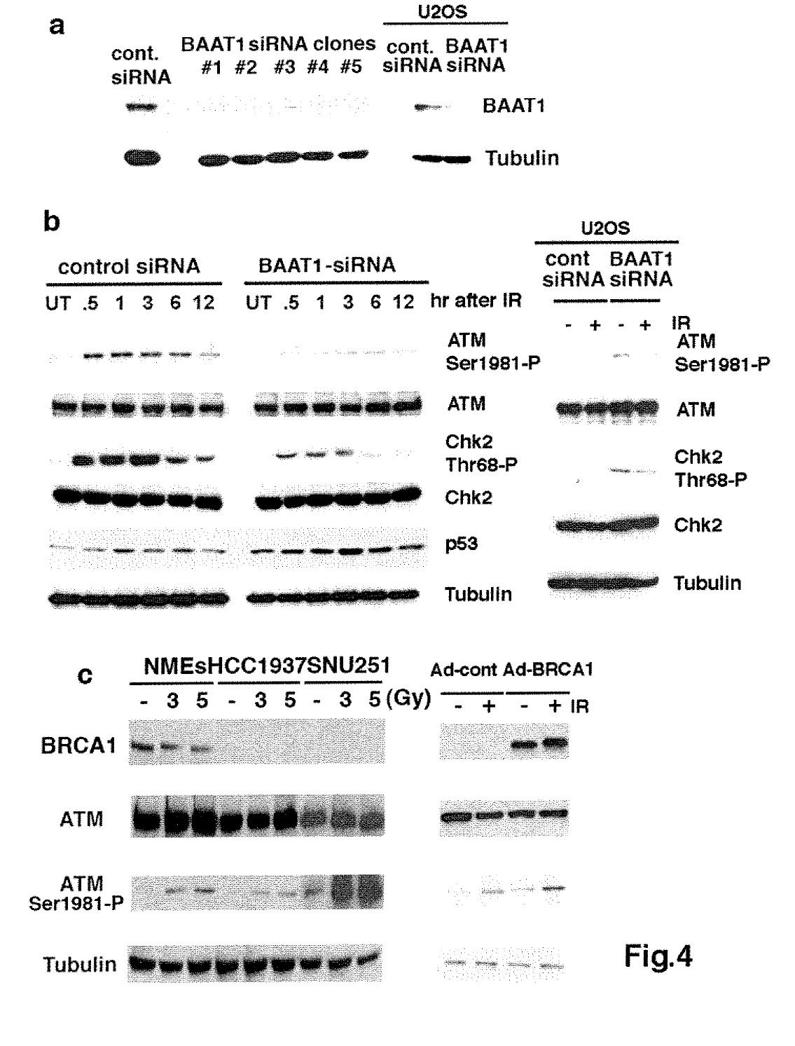

Functional interaction of BAAT1 with ATM was studied in human normal mammary epithelial cells (NMEs) after IR treatment. BAAT1 levels were found to increase after DNA damage and the amount of BAAT1 associated with ATM also increased (FIG. 2A, Left). Similarly, increased interaction between BRCA1 and BAAT1 after IR treatment (1 h) was confirmed by co-immunoprecipitation assay (FIG. 2A, Right). ATM's involvement in DSBs-signaling and localization of its substrate, γH2AX to DSB has been thoroughly studied (Bakkenist et al., “DNA Damage Activates ATM Through Intermolecular Autophosphorylation and Dimer Dissociation.” Nature 421:499-506 (2003); Shroff et al., “Distribution and Dynamics of Chromatin Modification Induced by a Defined DNA Double-Strand Break,” Curr Biol 14:1703-1711, which are hereby incorporated by reference in their entirety). Although no significant signals of γH2AX signals had been detected in NMEs before IR treatment, co-localization of BAAT1 and γH2AX was increased 1 h after treatment, demonstrating that BAAT1 is present in DSBs (FIG. 2B). Specificity of anti BAAT1 antibody for immunocytochemical analysis was confirmed with NMEs transfected with BAAT1-siRNA (FIG. 2B, bottom; FIG. 4A). These results demonstrate that BAAT1 is involved in the ATM-mediated DNA damage pathway.

### Example 4

### Co-Localization of BAAT1, BRCA1, and ATM

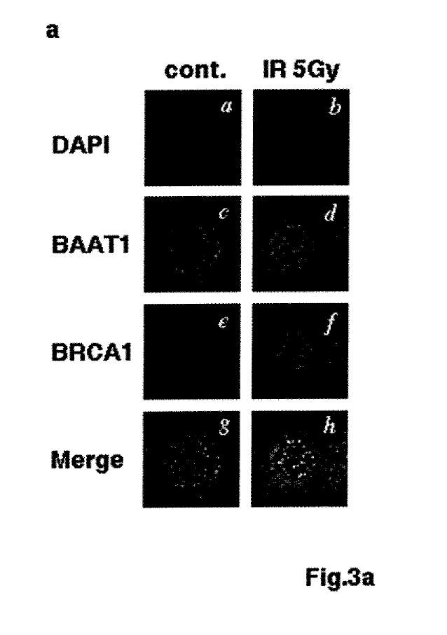

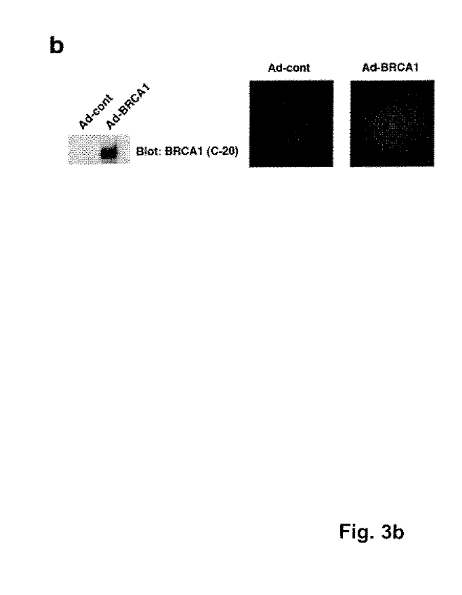

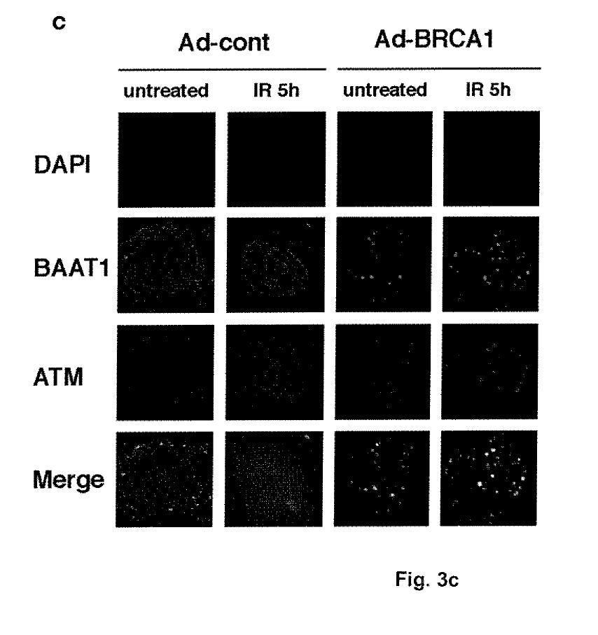

NMEs were immunostained with anti BRCA1 and BAAT1 antibodies under conditions of IR damage. Before IR treatment, both nucleus and cytoplasmic BAAT1 were detected (FIG. 3A, panels a and c) and co-localization with BRCA1 was not prominent (FIG. 3A, panels a and g). After IR treatment, however, overlapped signals increased in the nucleus (FIG. 3A, panels a and h). Of note, not all of BAAT1 was colocalized with BRCA1 in IR treated cells. Immunocytochemical analysis of BAAT1 and ATM was further studied using SNU251 cells, an ovarian cancer cell line carrying truncated BRCA1. SNU251 cells were infected with control or BRCA1 adenovirus for 48 h. BRCA1 expression was confirmed by immunoblot analysis and immunocytochemistry, which showed the nuclear dot pattern of the protein. Cells were then treated with IR and localization of BAAT1 and ATM was determined using specific antibodies after 5 h. Immunocytochemical analysis showed that nuclear and cytoplasmic ATM and BAAT1 in the control virus infected SNU251 cells. Although BRCA1 expression induced nuclear localization of ATM and BAAT1, no significant colocalization of both proteins was observed (FIG. 3C, bottom). However, co-localization of both proteins was increased after IR treatment in BRCA1 expressing cells (FIG. 3C, bottom). By counting BAAT1 foci detected by immunostaining in more than 50 cells, 55%, 42% and 44% of BAAT1 co-localized to BRCA1, γH2AX and ATM, respectively. These results demonstrate that co-localization of BAAT1 and ATM is BRCA1-dependent.

### Example 5

### BAAT1 is Required for ATM Ser¹⁹⁸¹ Phosphorylation Under Conditions of IR Damage

Recent studies have revealed that phosphorylation of Ser¹⁹⁸¹ of ATM protein is crucial for activation of its catalytic activity induced by DNA damage (Bakkenist et al., “DNA Damage Activates ATM Through Intermolecular Autophosphorylation and Dimer Dissociation.” Nature 421:499-506 (2003), which is hereby incorporated by reference in its entirety). The role of BAAT1 in regulation of ATM activation was explored by decreasing the levels of BAAT1 by siRNA under conditions of DNA damage. NMEs were stably transfected with the BAAT1-specific pSUPER construct (Brummelkamp et al., “A System for Stable Expression of Short Interfering RNAs in Mammalian Cells,” Science 296:550-553, which is hereby incorporated by reference in its entirety) and several clones were isolated (FIG. 4A, Left). Cells were treated with IR and phosphorylation of ATM was studied at 0.5 h, 1 h, 3 h, 6 h and 12 h after treatment. As shown in FIG. 4B, phosphorylation of Ser¹⁹⁸¹ was significantly impaired in BAAT1-knockdown cells after IR treatment. Consistent with this, phosphorylation of Thr68 of Chk2, a known ATM substrate, was also decreased in BAAT1-siRNA cells. Interestingly, depletion of BAAT1 increased p53 without IR treatment, which was slightly increased 3h after treatment (FIG. 4B). Impaired phosphorylation of ATM by IR treatment was also detected in U2OS cells transiently transfected with BAAT1 siRNA (FIG. 4A, Right, FIG. 4B, Right).

The requirement for BAAT1 in ATM activation was studied in BRCA1-mutated HCC1937 and SNU251 cells (FIG. 4C). After treatment of these cells with IR (3Gy or 5Gy), cells lysates were immunoblotted for ATM Ser¹⁹⁸¹ phosphorylation. Interestingly, ATM Ser¹⁹⁸¹ is phosphorylated in response to IR damage in both cell types. Interestingly, BRCA1 expression in SNU251 cells enhanced ATM phosphorylation by IR (FIG. 4C, Right). These results demonstrate that, although BRCA1 is not essential for ATM activation, it can enhance its phosphorylation.

### Example 6

### BAAT1 Depletion Causes Apoptosis without DNA Damage

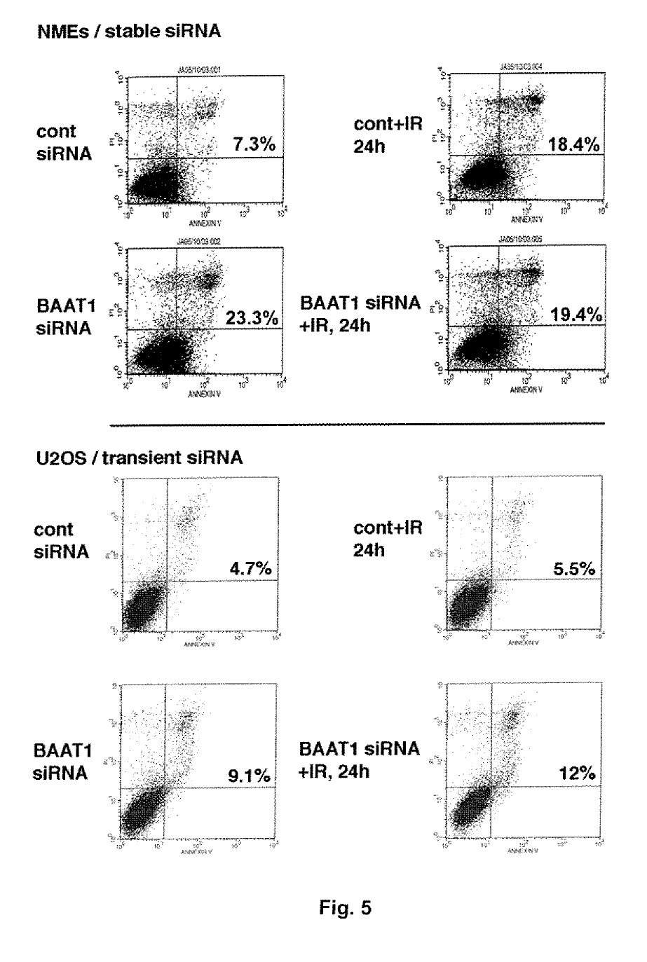

Because BAAT siRNA cells showed high levels of p53, it was examined whether those cells show any biological phenotype. Annexin V analysis revealed that BAAT1-siRNA cells showed constitutive apoptosis (23.3%) compared to control cells (7.3%) and IR treatment did not enhance this phenotype (19.4%) (FIG. 5, top). Similar phenotype was observed in U2OS cells transiently transfected with BAAT1 siRNA. These cells showed increased cell death by depletion of BAAT1 from 4.7% to 12%. These results demonstrate that BAAT1 is involved in the regulation of ATM phosphorylation after DNA damage and that loss of BAAT1 results in the activation of p53-mediated checkpoint, leading to apoptosis.

### Example 7

### Phosphorylation of ATM Substrates is Decreased in BAAT1 Knockdown Cells

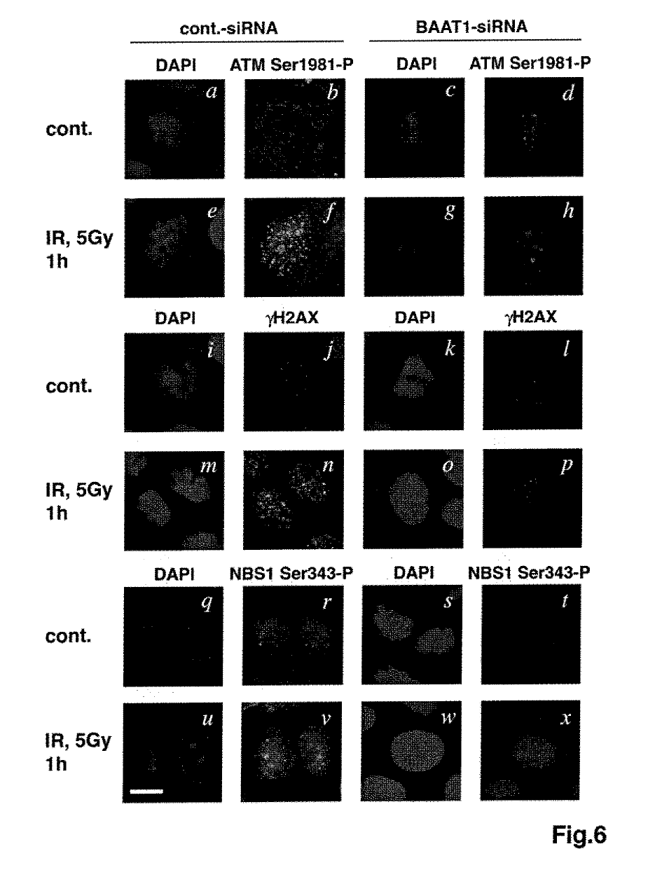

Impaired Ser¹⁹⁸¹ phosphorylation under conditions of DNA damage was also detected by immunocytochemical analysis. Although control cells showed increased nuclear signals of phosphorylated Ser¹⁹⁸¹ after IR treatment (FIG. 6,f), such signals were greatly reduced in BAAT1-siRNA cells (FIG. 6, h). Consistent with these results, phosphorylation of γH2AX and NBS1 was similarly impaired in BAAT1-siRNA cells after IR treatment (FIG. 6, n and p, v and x).

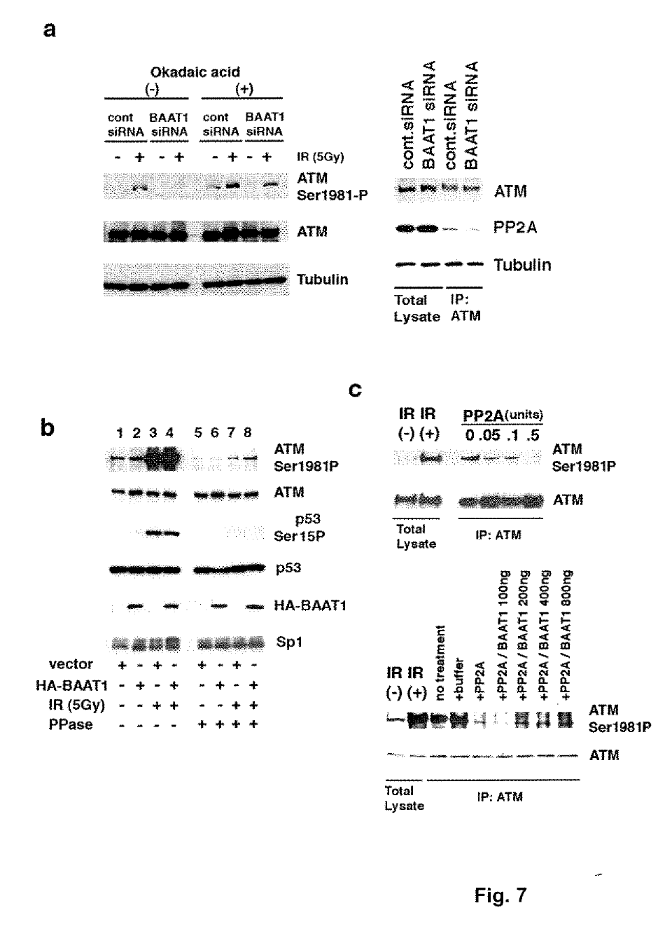

As one possible mechanism of impaired ATM phosphorylation in BAAT1-siRNA cells, loss of BAAT1 may result in increased activity of ATM phosphatase. Recently, it has been demonstrated that the protein phosphatase PP2A physically binds to ATM and regulates Ser¹⁹⁸¹ phosphorylation (Goodarzi et al., “Autophosphorylation of Ataxia-Telangiectasia Mutated is Regulated by Protein Phosphatase 2A,” EMBO J 23:4451-4461 (2004), which is hereby incorporated by reference in its entirety). In these studies, a phosphatase inhibitor, okadaic acid, was shown to induce autophosphorylation of ATM. On the basis of these results, possible roles of BAAT1 in ATM dephosphorylation were studied using okadaic acid in BAAT1-siRNA cells. Control and BAAT1-siRNA cells were treated with okadaic acid before IR treatment. Impaired phosphorylation of ATM Ser¹⁹⁸¹ in BAAT1-siRNA cells was markedly restored after okadaic acid treatment (FIG. 7A, Left). Interestingly, interaction between ATM and PP2A was not changed in BAAT1 siRNA cells (FIG. 7A, Right). Whether BAAT1 is involved in protection of the phosphorylated ATM was further examined. HA-tagged BAAT1 was expressed in 293T cells and cell lysates were treated with PP2A for 4 h at 37° C. As shown in FIG. 7B, expression of HA-tagged BAAT1 partially protected phosphorylatin of ATM Ser¹⁹⁸¹, but not p53Ser15, induced by IR. Protection of phosphorylated ATM from PP2A was further examined by in vitro phosphatase assay. ATM was immunoprecipitated from NMEs after IR, followed by incubation with PP2A in phosphatase buffer. Significantly, dephosphorylation of ATM Ser1981 was partially blocked in the presence of full length, baculovirus produced BAAT1 (FIG. 7C bottom). These results suggest that BAAT1 plays a role in the protection of Ser¹⁹⁸¹ phosphorylation of ATM from ATM-phosphatase under conditions of DNA damage.

### Discussion of Examples 1-7

Regulation of cellular responses to DNA damage is clearly important for the maintenance of genome stability. Many proteins are involved in the early stage of this pathway, such as BRCA1, ATM/ATR and Mre11/Rad50/NBS1 (MRN) complex (Cortez et al., “Requirement of ATM-Dependent Phosphorylation of BRCA1 in the DNA Damage Response to Double-Strand Breaks,” Science 286:1162-1166 (1999); Gatei et al, “Role for ATM in DNA Damage-Induced Phosphorylation of BRCA1,” Cancer Res 60:3299-3304 (2000); Uziel et al., “Requirement of the MRN Complex for ATM Activation by DNA Damage,” EMBO J22:5612-5621 (2003); Carson et al., “The Mre11 Complex is Required for ATM Activation and the G2/M Checkpoint,” EMBO J 22:6610-6620 (2003); Li et al., “Functional Link of BRCA1 and Ataxia Telangiectasia Gene Product in DNA Damage Response, Nature 406:210-215 (2000), which are hereby incorporated by reference in their entirety). Recent studies have demonstrated a complicated mechanism of ATM activation: alteration of the chromatin/chromosome structures directly affects its activation (Bakkenist et al., “DNA Damage Activates ATM Through Intermolecular Autophosphorylation and Dimer Dissociation.” Nature 421:499-506 (2003); Chun et al., “ATM Protein Purified from Vaccinia Virus Expression System: DNA Binding Requirements for Kinase Activation,” Biochem Biophys Res Commun 322:74-81 (2004), which are hereby incorporated by reference in their entirety), or MRN stimulates ATM activity by facilitating stable binding of substrates when NBS1 phosphorylation is present (Lee et al., “Direct Activation of the ATM Protein Kinase by the Mre11/Rad50/Nbs1 Complex.” Science 304:93-96 (2004); Lee et al., “ATM Activation by DNA Double-Strand Breaks Through the Mre11-Rad50-Nbs1 Complex.” Science 308:551-554 (2005), which are hereby incorporated by reference in their entirety). Thus, the MRN complex was found to be a sensor and effector of ATM activation and signaling in response to DSBs. Complementary results indicate that the NBS1 subunit serves as a bridge between ATM and the DNA-bound MR heterodimer (Falck et al., “Conserved Modes of Recruitment of ATM, ATR and DNA-PKcs to Sites of DNA Damage,” Nature 434:605-611 (2005), which is hereby incorporated by reference in its entirety). Although the original model of ATM activation involved transmission of the DNA damage signal to ATM through structural changes in chromatin, recent studies suggest another model in which the MRN complex forms a bridge between ATM and DSBs sites and delivers the signal that triggers ATM Ser1981 autophosphorylation and monomer formation. Of interest, a pathway involving 53BP1 may sense DSBs. 53BP1 was recently identified as a sensor of DSBs through a mechanism involving unstacking of nucleosomes at sites of DNA DSBs that expose a 53BP1-binding site on histone H3 (Huyen et al., “Methylated Lysine 79 of Histone H3 Targets 53BP1 to DNA Double-Strand Breaks,” Nature 432:406-411 (2004), which is hereby incorporated by reference in its entirety). The evidence indicates that 53BP1 and MRN activate ATM through distinct pathways (Mochan et al., “53BP1 and NFBD1/MDC1-Nbs1 Function in Parallel Interacting Pathways Activating Ataxia-Telangiectasia Mutated (ATM) in Response to DNA Damage,” Cancer Res 63:8586-8591 (2003), which is hereby incorporated by reference in its entirety). Furthermore, 53BP1 is recruited to chromatin in cells treated with hypotonic media (Huyen et al., “Methylated Lysine 79 of Histone H3 Targets 53BP1 to DNA Double-Strand Breaks,” Nature 432:406-411 (2004), which is hereby incorporated by reference in its entirety), but ATM activation does not require NBS1 under such conditions (Difilippantonio et al., “Role of Nbs1 in the Activation of the ATM Kinase Revealed in Humanized Mouse Models,” Nat Cell Biol 7:675-685 (2005), which is hereby incorporated by reference in its entirety). Although current results support the notion that assembly of BRCA1/ATM/BAAT1 is crucial for ATM activation after IR treatment, it remains to be determined whether BAAT1 is involved in MRN pathways. However, co-localization and co-immunoprecipitation of Mre11 and BAAT1 was observed, suggesting that at least a certain fraction of BAAT1 may participate in the MRN pathway. It was found that in BAAT1-siRNA cells, ATM phosphorylation is significantly decreased after IR treatment. There are several possibilities to explain this finding: (a) ATM is not recruited to the sites of DSBs; (b) ATM is recruited to the sites of DSBs but it remains in its dimeric form; and/or (c) activity of ATM phosphatase is increased in IR-treated cells. It has been shown recently that the protein phosphatase PP2A interacts with ATM in undamaged cells and that IR induces phosphorylation-dependent dissociation of PP2A from ATM (Goodarzi et al., “Autophosphorylation of Ataxia-Telangiectasia Mutated is Regulated by Protein Phosphatase 2A,” EMBO J 23:4451-4461 (2004), which is hereby incorporated by reference in its entirety). The investigators also showed that the PP2A inhibitor okadaic acid induces autophosphorylation of ATM on Ser¹⁹⁸¹ in undamaged cells. It was found that okadaic acid treatment restores ATM autophosphorylation induced by IR in BAAT1-siRNA cells. Since okadaic acid treatment does not induce γH2AX foci, it is likely that it induces ATM autophosphorylation by inactivation of a protein phosphatase rather than by inducing DSBs (Goodarzi et al., “Autophosphorylation of Ataxia-Telangiectasia Mutated is Regulated by Protein Phosphatase 2A,” EMBO J 23:4451-4461 (2004), which is hereby incorporated by reference in its entirety). The physical interaction between ATM and PP2A in BAAT1-siRNA cells was examined, however, no significant changes in their interaction were detected. These results suggest that, although BAAT1 is involved in the regulation of ATM phosphatase, a precise role of the protein in this pathway remains to be elucidated. To understand more complex nuclear events, further investigation should focus on the specificity of BAAT1 function under various stimuli that activates ATM activity. The generation of mice carrying a mutant BAAT1 locus should provide definitive evidence of BAAT1's physiological roles in the DNA damage pathway.

### Example 8

### Characterization of the Role of BAAT1 in DNA Damage Response Signaling through the Generation of BAAT1 Mutant Mice

The role of BAAT1 in DNA damage response signaling through the generation of BAAT1 mutant mice will be characterized. Previous in vitro experiments support the view that BAAT1 regulates ATM function. Further functional experiments will be performed to determine the role of BAAT1 in ATM signaling in vivo. Preliminary studies of the ATM-BAAT1 interaction have shown that loss of BAAT1 results in the abrogation of ATM activation under the conditions of IR damage in human cells, consistent with the conclusion that BAAT1 is a positive regulator of ATM. Thus, the hypothesis is that loss of this signaling pathway may increase DSB or result in the delayed DSB repair in vivo. It is expected that elimination of BAAT1 function by gene-knockout in mice would make it possible to further investigate the physiological roles of BAAT1/ATM/BRCA1 complex in DNA damage pathway. Although such an experiment does not prove that ATM is the only BAAT1 target that contributes to the regulation of cell radiosensitivity, it certainly adds correlative evidence to support this view. Chimeric BAAT1 mice have been generated and homozygous mice are in the process of being generated. Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of screening for potential therapeutics that promote a DNA damage response, said method comprising:
providing a BAAT1 protein;
providing an ataxia telangiectasia mutated;
providing a candidate compound;
combining the BAAT1 protein, the ataxia telangiectasia mutated, and the candidate compound in a test mixture under conditions suitable for the ataxia telangiectasia mutated to undergo phosphorylation;
evaluating whether the candidate compound increases phosphorylation levels of the ataxia telangiectasia mutated compared to when the candidate compound is not present in the test mixture; and
identifying a candidate compound that increases phosphorylation levels of ataxia telangiectasia mutated compared to when the candidate compound is not present in the test mixture as a potential therapeutic that promotes a DNA damage response.

2. The method according to claim 1, wherein the test mixture further comprises a BRCA1 gene.

3. The method according to claim 1, wherein said evaluating examines phosphorylation of ataxia telangiectasia mutated at serine 1981.

4. The method according to claim 1, wherein the BAAT1 protein has an amino acid sequence of SEQ ID NO: 1 or SEQ ID NO:2.

5. The method according to claim 1, wherein phosphorylation activates ataxia telangiectasia mutated.

6. A method of treating DNA damage in a subject, said method comprising:
administering a compound that increases phosphorylation levels of ataxia telangiectasia mutated under conditions effective to treat DNA damage.

7. A method of ascertaining changes in DNA damage status in a subject, said method comprising:
providing a sample from the subject;
determining ataxia telangiectasia mutated phosphorylation levels in the sample; and
comparing ataxia telangiectasia mutated phosphorylation levels in different samples taken from the subject at different times to ascertain changes in DNA damage status in the subject.

8. The method according to claim 7, wherein said determining comprises:
providing a BAAT1 protein;
providing an ataxia telangiectasia mutated;
combining the BAAT1 protein, the ataxia telangiectasia mutated, and the sample in a test mixture under conditions suitable for the ataxia telangiectasia mutated to undergo phosphorylation; and
evaluating whether the phosphorylation level of the ataxia telangiectasia mutated has changed.

9. The method according to claim 8, wherein the test mixture further comprises a BRCA1 gene.

10. The method according to claim 7, wherein said determining examines phosphorylation of ataxia telangiectasia mutated at serine 1981.

11. The method according to claim 8, wherein the BAAT1 has an amino acid sequence of SEQ ID NO: 1 or SEQ ID NO:2.

12. The method according to claim 7, wherein phosphorylation activates ataxia telangiectasia mutated.

13. The method according to claim 7, wherein the sample is a bodily fluid.

14. The method according to claim 13, wherein the bodily fluid is urine or blood.

15. The method according to claim 7, wherein the sample is tissue.

16. A method of screening for potential therapeutics for treating breast cancer, said method comprising:
providing a BAAT1 protein;
providing a BRCA1 protein;
providing a candidate compound;
combining the BAAT1 protein, the BRCA1 protein, and the candidate compound in a test mixture under conditions suitable for the BAAT1 protein to bind to the BRCA1 protein;
evaluating whether the candidate compound increases binding of the BAAT1 protein to the BRCA1 protein; and
identifying a candidate that increases binding of the BAAT1 protein to the BRCA1 protein as a potential therapeutic for treating breast cancer.

17. The method according to claim 16, wherein the BAAT1 protein has an amino acid sequence of SEQ ID NO: 1 or SEQ ID NO:2.

18. A method of treating breast cancer in a subject, said method comprising:
administering a compound to the subject which is effective in promoting binding of BAAT1 protein to BRCA1 protein under conditions effective to treat breast cancer.

19. The method according to claim 18, wherein the BAAT1 protein has an amino acid sequence of SEQ ID NO: 1 or SEQ ID NO:2.

20. A method of detecting whether a subject is susceptible to breast cancer, said method comprising:
providing a sample from the subject;
determining a level of binding of BRCA1 protein to BAAT1 protein binding in the sample; and
comparing the level of binding of BRCA1 protein to BAAT1 protein in the sample to a standard level of binding of BRCA1 protein to BAAT1 protein for an individual that does not have breast cancer and/or for an individual that does have breast cancer; and
identifying whether the subject is susceptible to breast cancer based on how the level of level of binding of BRCA1 protein to BAAT1 protein in the sample correlates to the standard level.

21. The method according to claim 20, wherein said determining comprises:
providing a BAAT1 protein;
providing a BRCA1 protein;
combining the BAAT1 protein, the BRCA1 protein, and the sample in a test mixture under conditions suitable for the BAAT1 protein and the BCRA1 to bind; and
evaluating the binding level of BRCA1 and BAAT1 in the test mixture.

22. The method according to claim 20, wherein the BAAT1 has an amino acid sequence of SEQ ID NO: 1 or SEQ ID NO:2.

23. The method according to claim 20, wherein the sample is a bodily fluid.

24. The method according to claim 23, wherein the bodily fluid is urine or blood.

25. The method according to claim 20, wherein the sample is tissue.


SEQUENCE LISTING (5 sequences)

SEQ ID NO: 1 - protein, 821 aa, Homo sapiens
MDPECAQLLPALCAVLVDPGQPVADDTCLEKLLDWFKTVTEGESSVVLLQEHPCLVELLSHVLKVQDLSSGVLSFSLRLAGTFAAQENCFQYLQQGELLPGLFGEPGPLGRATWAVPTVRSGWIQGLRSLAQHPSALRFLADHGAVDTIFSLQGDSSLFVASAASQLLVHVLALSMRGGAEGQPCLPGGDWPACAQKIMDHVEESLCSAATPKVTQALNVLTTTFGRCQSPWTEALWVRLSPRVACLLERDPIPAAHSFVDLLLCVARSPVFSSSDGSLWETVARALSCLGPTHMGPLALGILKLEHCPQALRTQAFQVLLQPLACVLKATVQAPGPPGLLDGTADDATTVDTLLASKSSCAGLLCRTLAHLEELQPLPQRPSPWPQASLLGATVTVLRLCDGSAAPASSVGGHLCGTLAGCVRVQRAALDFLGTLSQGTGPQELVTQALAVLLECLESPGSSPTVLKKAFQATLRWLLSSPKTPGCSDLGPLIPQFLRELFPVLQKRLCHPCWEVRDSALEFLTQLSRHWGGQADFRCALLASEVPQLALQLLQDPESYVRASAVTAMGQLSSQGLHAPTSPEHAEARQSLFLELLHILSVDSEGFPRRAVMQVFTEWLRDGHADAAQDTEQFVATVLQAASRDLDWEVRAQGLELALVFLGQTLGPPRTHCPYAVALPEVAPAQPLTEALRALCHVGLFDFAFCALFDCDRPVAQKSCDLLLFLRDKIASYSSLREARGSPNTASAEATLPRWRAGEQAQPPGDQEPEAVLAMLRSLDLEGLRSTLAESSDHVEKSPQSLLQDMLATGGFLQGDEADCY

SEQ ID NO: 2 - protein, 822 aa, Mus musculus
MDPECSRLLPALCAVLADPRQLVADDTCLEKLLDWFKTVTEAESSLQLLQDHPCLMELLSHVLKPQDVSPRVLSFALRLVGVFAAQEDCFEYLQQGELLLGLFGESGAPGWAAWSIPSVRSGWIQGLCYLAHHPSALHFLADSGAVDTLFSLQGDPSLFVASAASQLLVHILALSMQGGAPGSPVPEAAAWPMCAQKIVNHVDESLHAKATPQVTQALNVLTTTFGRCHNPWTGVLWERLSPPVARLFERDPIPAVHALMDLLLSVARSPVLNFAACGLWEMLAQTLSRLSPIQAGPLALGTLKLQHCPQELRTQAFGVLLQPLACILKATTQAPGPPGLLDGTVGSLLTVDILLASKSACVGLLCQTLAHLEELQMLPQCPSPWPQVHLLQAALTILHLCDGSADPSSSAGGRLCGTLGGCVRVQRAALDFLGTLSQGTSPLELVLEVFAVLLKTLESPESSPMVLKKAFQATLRWLQNPHKTPSSSDLSSDALLFLGELFPILQKRLCSPCWEVRDSALEFLTHLIRHWGGQADFREALRSSEVPTLALQLLQDPESYVRASAVGAAGQLSSQGLQAAPASPENSQAQQGLLMDLMHILSTDSEGFPRRAVLRVFTDWLRDGHADVVRDTEWFVATVLQAVSRDLDWEVRVQGLELARVFLTQALGQPSLHCPYTVGLPRASSPRPHPEFLQTLCRLPLFEFAFCALLDCDRPVAQKACDLLLFLRDKTVPCSSPREAGDSPNSASVEAALQRWREGEQAQPLGDLDPEAMLAILRALDLEGLQGRLAKSSDHVEKSPQSLLQDMLATVGVLEENEADCY

SEQ ID NO: 3 - protein, 17 aa, Artificial
  peptide
AESSDHVEKSPQSLLQD

SEQ ID NO: 4 - RNA, 18 nt, Artificial
  synthetic
aacggucacugaaggaga

SEQ ID NO: 5 - DNA, 19 bp, Artificial
  synthetic
gttacgctgagtacttcga